(19) United States
(12) Patent Application Publication
(10) Pub. No.: US 2018/0250496 A1
(43) Pub. Date: Sep. 6, 2018

(54) RAPID EXCHANGE MULTIPLE CATHETER SYSTEM

(71) Applicant: COOK MEDICAL TECHNOLOGIES LLC, Bloomington, IN (US)

(72) Inventors: Jeffry S. Melsheimer, Springville, IN (US); Alex Broderick, Bloomington, IN (US); Melissa L. Dale, Linton, IN (US); David Gordon, Bloomington, IN (US); Marshall A. Lord, New Braunfels, TX (US)

(73) Assignee: COOK MEDICAL TECHNOLOGIES LLC, Bloomington, IN (US)

(21) Appl. No.: 15/909,623

(22) Filed: Mar. 1, 2018

Related U.S. Application Data
(60) Provisional application No. 62/466,543, filed on Mar. 3, 2017.

(51) Int. Cl. A61M 25/00; A61M 25/01; A61B 1/04

(52) U.S. Cl. CPC A61M 25/0029; A61M 25/0105; A61B 1/018; A61M 2025/0183; A61B 1/042

(57) ABSTRACT

A multiple catheter system including an imaging scope having an elongated body having a first length and a first outer diameter. The elongated body including a first passage extending along the first length. A flexible distal tip portion is coupled to the elongated body. The flexible distal tip portion includes an imaging sensor and a light source. A proximal connector is coupled to the elongated body opposite the flexible distal tip portion. The proximal connector has a second outer diameter not greater than the first outer diameter. A first catheter includes a flexible distal tip portion and a first lateral extension disposed at the flexible distal tip portion. The first lateral extension includes a second passage having an inner diameter greater than the first outer diameter and the second outer diameter, wherein the imaging scope is movably positionable within the second passage.

### CROSS-REFERENCE TO RELATED APPLICATION

This invention claims the benefit of priority of U.S. Provisional Application No. 62/466,543, entitled “Rapid Exchange Multiple Catheter System,” filed Mar. 03, 2017, which is hereby incorporated by reference in its entirety.

### BACKGROUND

Removal of kidney stones and other calculi within body cavities may be accomplished with an endoscope. The endoscope is inserted into the patient through a body passageway, such as the ureter. The endoscope includes an integral optical system, a working channel, and a controller to maneuver the endoscope so that the surgeon can accomplish a therapeutic or diagnostic procedure. The surgeon positions the endoscope so that the surgeon can observe the desired body part of the patient using the optical system, with irrigation if necessary. The surgeon then uses at least one instrument, such as a laser or a grasper, to break up and remove objects in the body passageway. The endoscope may also be used for diagnostic purposes, such as for observing the desired portion of the patient and then taking a biopsy sample.

Obstructions or blockages may be found during observations of a patient's kidney. It is generally beneficial to remove these obstructions or blockages during the observation with minimal device change-outs. While visualization capabilities are important; it is also important to have the ability to deflect or steer the catheter to aid in navigation, and manipulate and/or actively diminish and remove the obstruction or blockage. Catheters designed to perform all these functions are typically bulky and expensive.

Effective diagnostic visualization, particularly, in small passages or spaces, before and/or during endoscopic surgery including an increased ability to navigate through tortuous body passageways and cavities while allowing for important access functions continues to be a priority.

### SUMMARY

In one example embodiment, an imaging scope for a multiple catheter system includes an elongated body having a first length. The elongated body includes a passage extending along the first length. A flexible distal tip portion is coupled to the elongated body. An imaging device including a wiring harness at least partially disposed in the passage and an imaging sensor is disposed at the flexible distal tip portion. The imaging sensor is operatively coupled to the wiring harness. A proximal portion is coupled to the elongated body opposite the flexible distal tip portion. The proximal portion has an outer surface. The proximal portion includes a proximal termination of the wiring harness, e.g., a printed circuit board (PCB), operatively coupled to the imaging sensor. An annular slip ring is electrically coupled to the proximal termination.

In another example embodiment, a multiple catheter system includes an imaging scope having an elongated body having a first length and a first outer diameter. The elongated body includes a first passage extending along the first length. A flexible distal tip portion is coupled to the elongated body. The flexible distal tip portion includes an imaging sensor and a light source. A proximal connector is coupled to the elongated body opposite the flexible distal tip portion. The proximal connector is operatively coupled to the imaging sensor and the light source. The proximal connector has a second outer diameter not greater than the first outer diameter. A first catheter, e.g., a steering catheter, includes a flexible distal tip portion and a first lateral extension disposed at the flexible distal tip portion. The first lateral extension forms a second passage having an inner diameter greater than the first outer diameter and the second outer diameter, wherein the imaging scope is movably positionable within the second passage.

In another example embodiment, a method for introducing an imaging scope into a lumen of a human includes slidably positioning a first catheter about a proximal portion of an imaging scope. The first catheter is advanced along a length of the imaging scope. A flexible distal tip portion of the first catheter is positioned proximal to a distal portion of the imaging scope. The first catheter and the imaging scope can be advanced through the lumen to a target site.

### BRIEF DESCRIPTION OF THE DRAWINGS

The detailed description is set forth with reference to the accompanying figures. The use of the same reference numbers in different figures indicates similar or identical items or features.

FIG. 1 is a perspective view of a distal portion of an example multiple catheter system, according to various embodiments;

FIG. 2 is a perspective view of a distal portion of an example multiple catheter system, according to various embodiments;

FIG. 3 is a sectional view of a distal portion of an example multiple catheter system, according to various embodiments;

FIG. 4 is a partial sectional side view of an example multiple catheter system, according to various embodiments;

FIG. 5 is a side view of an example imaging scope for a multiple catheter system, according to various embodiments;

FIG. 6 is a side view of a distal portion of an example imaging scope positioned within a passage of a first catheter of a multiple catheter system, according to various embodiments;

FIG. 7 is a perspective view of a proximal portion of an example imaging scope, according to various embodiments;

FIG. 8 is a perspective view of an imaging control unit, according to various embodiments;

FIG. 9 is a perspective view of the imaging control unit of FIG. 8 in an open configuration, according to various embodiments;

FIG. 10 is a perspective view of a portion of an interior surface of the imaging control unit of FIG. 8, according to various embodiments;

FIG. 11 is a perspective view of a proximal connector of an example imaging scope, according to various embodiments;

FIG. 12 is a sectional side view of a distal portion of an example first catheter of a multiple catheter system, according to various embodiments;

FIG. 13 is a sectional side view of a distal portion of an example second catheter of a multiple catheter system, according to various embodiments;

FIG. 14 is a side view of a portion of an example multiple catheter system, according to various embodiments; and

FIG. 15 illustrates an example method for introducing an imaging scope into a lumen of a human, according to various embodiments.

### DETAILED DESCRIPTION

Example embodiments of the present invention are disclosed herein. It is understood, however, that the disclosed embodiments are merely exemplary and may be embodied in various and alternative forms. The figures are not necessarily to scale; some figures may be configured to show the details of a particular component. Therefore, specific structural and functional details disclosed herein are not to be interpreted as limiting but merely as a representative basis for the claims and/or teaching one skilled in the art to practice the embodiments.

Example embodiments seek to overcome some of the concerns associated with visualization of body pathways and cavities, which may be tortuous, during endoscopic and laparoscopic surgery. Example embodiments of a multiple catheter system are described that utilize “rapid-exchange” features to enable two or more devices to couple together, or to be selectively withdrawn while minimizing a profile of the multiple catheter system. For example, components may be replaced without discarding components that are still viable. Different size components may be mixed-and-matched. Further, a steerable component may be rotated independently of an imaging component so that visualization does not suffer during navigation. Because the components are not coaxial or fully nested or enveloped, the profile can be minimized.

An example embodiment of a multiple catheter system includes a first component, e.g., an imaging scope having a fiber optic or digital endoscope with a light source. The imaging scope has a proximal connector having a diameter equal to or less than a diameter of an elongated body of the imaging scope, such that other devices, e.g., a second component and/or a third component, can be moved over the proximal connecter and be maintained over the elongated body of the imaging scope with the imaging device acting as a wireguide. The second component, e.g., a steerable or deflectable first catheter, includes a control unit having a deflection actuator configured to allow the surgeon to control deflection of at least a distal portion of the first catheter. The distal portion of the first catheter includes a rapid-exchange feature, e.g., a first lateral extension, such as a tab, a collar, a projection, a protuberance, an appendage, or other suitable structure, made of a suitable material such as an extruded polymer, including a passage sized to accommodate the elongated body of the imaging device. The first catheter may also include a lumen or channel extending at least a portion of a length of the first catheter that can be used to introduce instruments or devices for suction, aspiration, and/or flushing, for example. A third component, e.g., a second catheter having a basket or snare at its distal portion, includes a rapid-exchange feature near its distal portion. In certain embodiments, the rapid-exchange feature of the second catheter is positioned proximal to the rapid-exchange feature of the first catheter with the first catheter and the second catheter coupled to the imaging device. In certain embodiments, these rapid-exchange features are similar, e.g., having the same or similar characteristics. The basket or snare disposed at the distal portion of the second catheter is movably positioned in a channel of the second catheter and extendible from the channel. The basket or snare is controlled by a controller disposed at a proximal portion of the second catheter. The second catheter may also include an additional lumen or channel extending at least a portion of a length of the second catheter that can be used to introduce instruments or devices for suction, aspiration, and/or flushing, for example. In a particular embodiment, the second catheter includes a laser, a lithotripsy device, or another suitable device for breaking and diminishing stones.

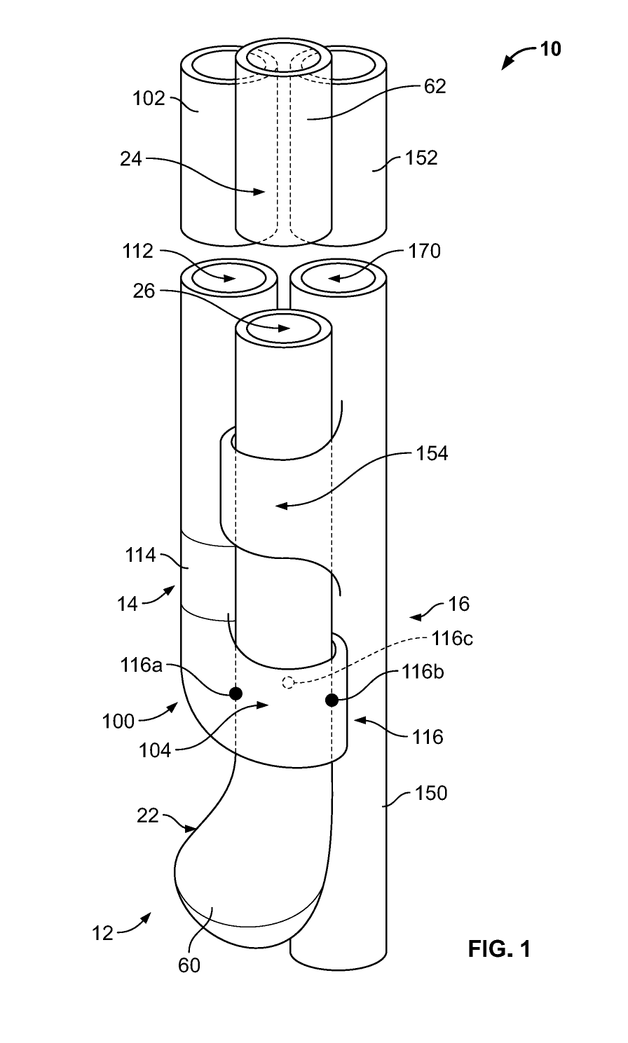

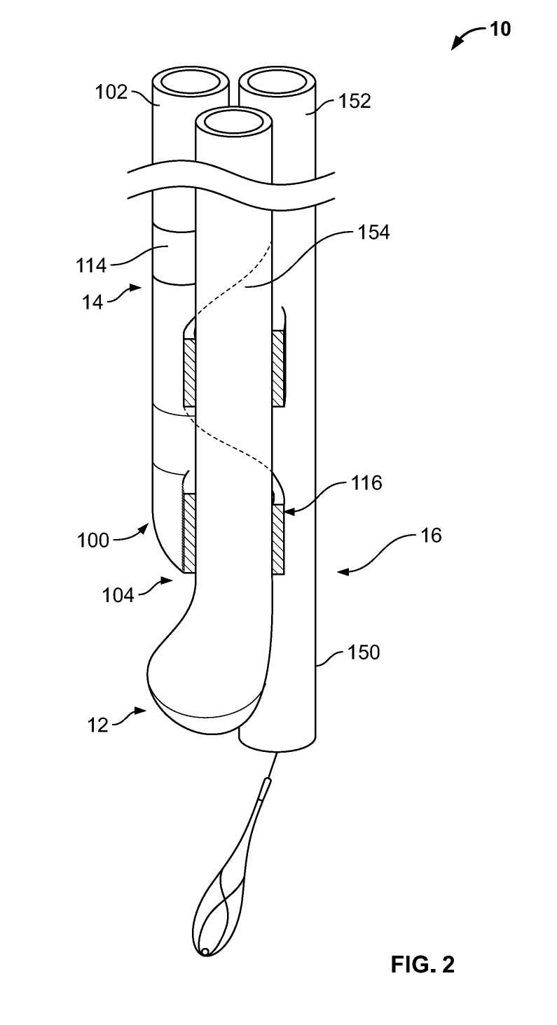

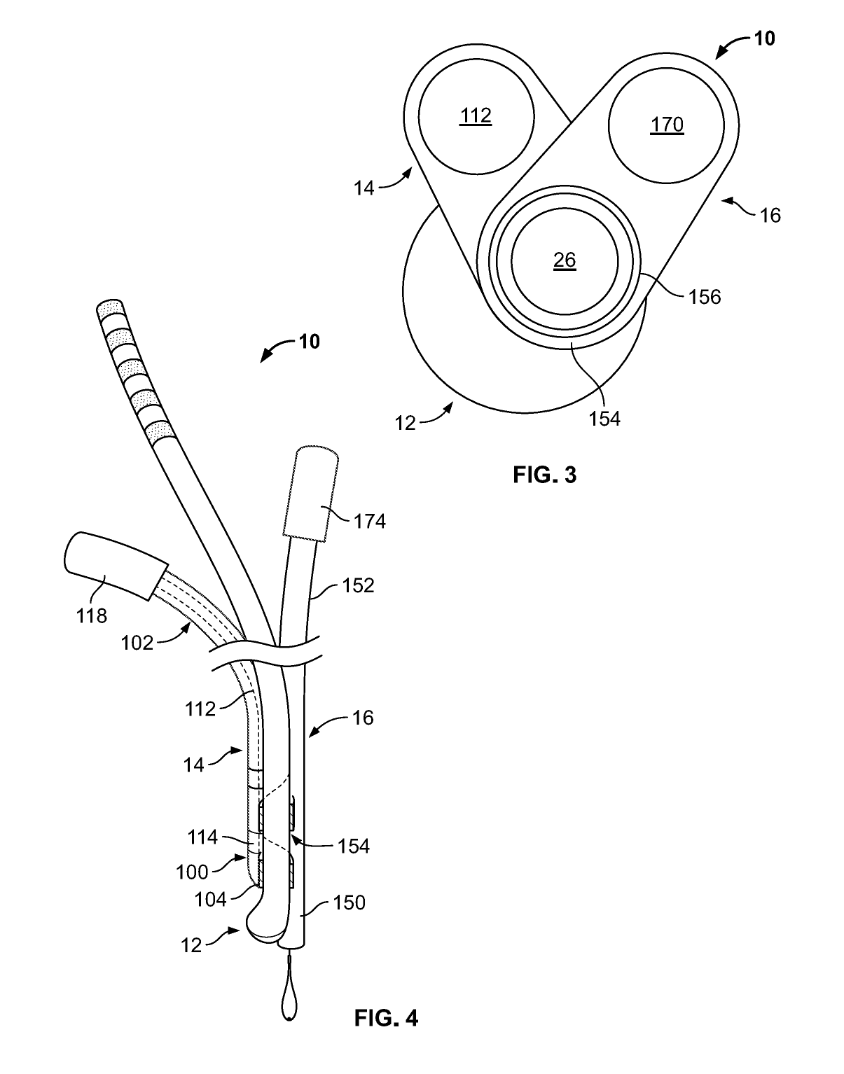

Referring now to the figures, FIGS. 1 and 2 are perspective views of a distal portion of an example multiple catheter system 10 including an imaging scope 12, a first catheter 14, such as a steerable or deflectable catheter capable of being steered or deflected through a lumen, for example, removably coupled to imaging scope 12, and a second catheter 16, such as a snare or basket catheter, for example, removably coupled to imaging scope 12, according to example embodiments. FIG. 3 is a sectional view of a distal portion of multiple catheter system 10, as shown in FIGS. 1 and 2. FIG. 4 is a partial sectional side view of multiple catheter system 10, shown in FIGS. 1 and 2. As shown in FIGS. 1 and 2, in example embodiments, first catheter 14 is positioned distally to second catheter 16 with first catheter 14 and second catheter 16 each movably positioned about imaging scope 12. Referring further to FIGS. 3 and 4, with first catheter 14 and second catheter 16 coupled to imaging scope 12, as described herein, an overall profile, e.g., outer circumference of multiple catheter system 10, can be minimized to facilitate movement of multiple catheter system 10 through a patient's lumen.

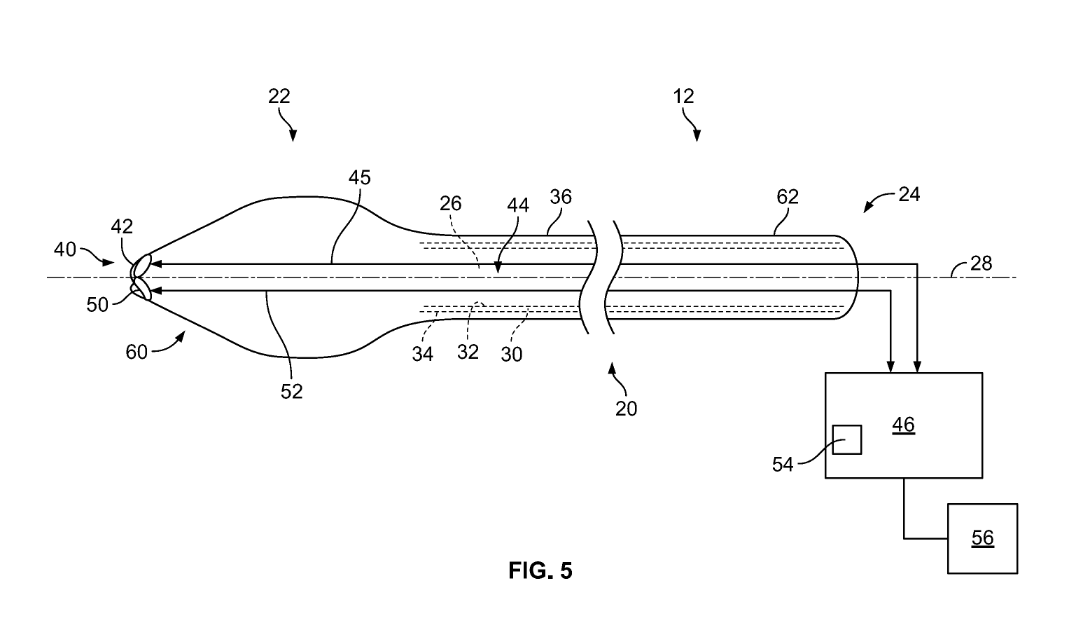

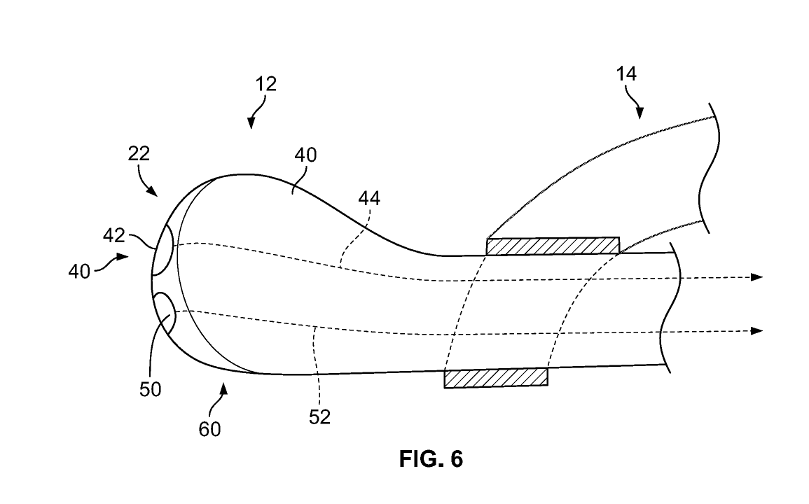

FIG. 5 is a side view of an example imaging scope 12 for multiple catheter system 10 and FIG. 6 is a side view of a distal portion of imaging scope 12 positioned within first catheter 14 of multiple catheter system 10, according to example embodiments. Referring to FIGS. 5 and 6, in example embodiments, imaging scope 12 includes an elongated body 20 having a distal portion 22 and an opposing proximal portion 24. Elongated body 20 has a first length that extends between distal portion 22 and proximal portion 24. Elongated body 20 includes at least one passage, such as passage 26 shown in FIG. 5, for example, which extends the first length of elongated body 20 along a longitudinal axis 28 of elongated body 20. In a particular embodiment, elongated body 20 includes a first passage, e.g., passage 26, and a second passage (not shown) each extending along the first length.

Referring further to FIG. 5, in one embodiment, elongated body 20 includes a core 30 having an inner surface 32 forming passage 26 and an outer surface 34 forming an outer surface of core 30. Core 30 provides rigidity and torque to imaging scope 12 to allow for increased control of navigation, particularly in tighter spaces. In example embodiments, core 30 is formed of a coil made of a suitable material, such as stainless steel, nitinol, nylon monofilament or other suitable plastic materials, in a coiled, braided or knit pattern, for example. As shown in FIG. 5, a sheath 36 is positioned about outer surface 34 of core 30. In example embodiments, sheath 36 has a hydrophilic outer surface to facilitate movement of imaging scope 12 within a lumen of the patient. For example, sheath 36 may be made of a suitable lubricious polymer material including, without limitation, a fluoropolymer liner, e.g., as polytetrafluoroethylene (PTFE) or Teflon® material, or another lubricious material such as polyethylene, polypropylene, nylon or polyurethane, for smooth movement of imaging scope 12 in the lumen during introduction of imaging scope 12 into the lumen and removal of imaging scope 12 from the lumen. Sheath 36 also protects the internal imaging device and the light source of imaging scope 12, as described below.

In example embodiments, elongated body 20 has a first length of 25.00 inches (63.50 centimeters (cm)) to 50.00 inches (127.00 cm), and, more particularly, a first length of 28.00 inches (71.12 cm) to 45.00 inches (114.30 cm), and, even more particularly, a first length of 29.537 inches (75.0 cm) to 39.370 inches (100.0 cm), suitable to allow the user to reach the multiple poles of the patient's kidney by transurethral introduction, for example. In alternative embodiments, elongated body 20 may have any suitable length less than 25.0 inches or greater than 50.00 inches. Elongated body 20 has an outer diameter of 0.010 inch (0.03 cm) to 0.118 inch (0.3 cm), and, more particularly, an outer diameter of 0.014 inch (0.0356 cm) to 0.063 inch (0.1400 cm). In a particular embodiment, elongated body 20 is tapered along at least a portion of the first length.

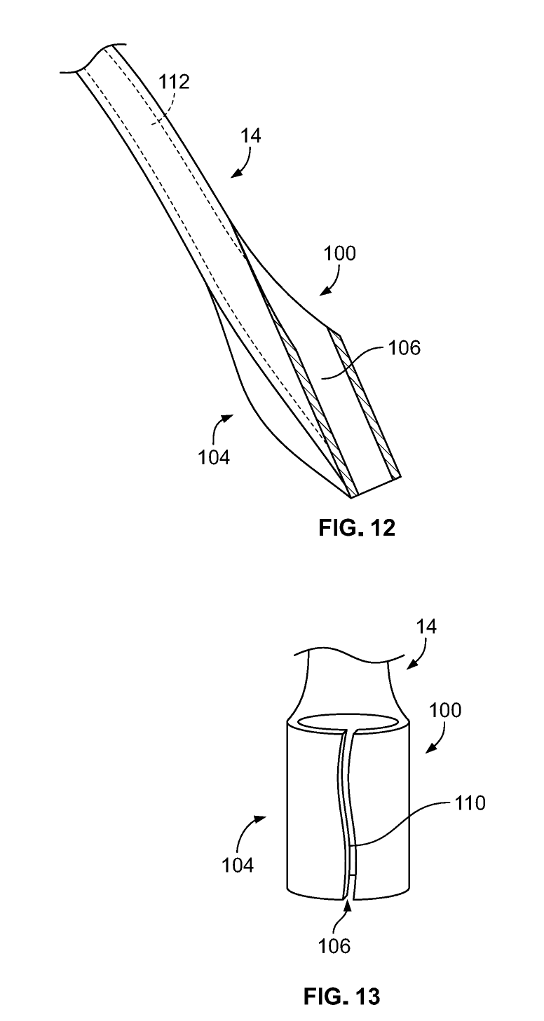

In example embodiments, an imaging device 40 extends through passage 26. Imaging device 40 includes an imaging sensor 42 disposed at distal portion 22 of elongated body 20 and a wiring harness 44 including one or more signal transmission connections 45 coupled to imaging sensor 42. As shown in FIG. 5, in certain embodiments, passage 26 through elongated body 20 is configured to accommodate at least a portion of imaging device 40. More specifically, at least a portion of wiring harness 44, e.g., signal transmission connection 45, extends through passage 26 to operatively couple, e.g., in signal or electronic communication, imaging sensor 42 to an imaging control unit 46, as shown in FIGS. 11 and 12, as described below, disposed at proximal portion 24 of elongated body 20 or to another suitable external processing system communicatively coupled to imaging sensor 42 through signal transmission connection 45. Imaging sensor 42 is configured to detect image information and transmit one or more signals indicative of the detected image information to signal transmission connection 45 and signal transmission connection 45 is configured to transmit the one or more signals indicative of the detected image information from imaging sensor 42 to imaging control unit 46.

In example embodiments, imaging device 40 includes a suitable imaging device sized and configured to navigate the tortuous passages and multiple poles of the patient's kidney by transurethral introduction. Imaging device 40 may include, for example, a solid state imaging device (SSID), such as a charged coupled device (CCD) camera, having a gradient refractive index (GRIN) lens. The term “solid state imaging device” generally refers to a camera or imaging device having a size approximately equal to or less than the diameter of a bundle of optical fibers. Suitable SSIDs include, for example, charge-injection devices (CID), charge-coupled devices (CCD), complementary metal oxide semiconductor (CMOS) devices, and other miniature-sized imaging devices, including those made from compound semiconductors such as InGaAs, capable of imaging reflected illumination of visible and/or non-visible light. In certain embodiments, the SSID is configured to transmit recorded images to imaging control unit 46 or another external processing system via signal transmission connection 45, disposed within passage 26. In alternative embodiments, the image information is sent via a wireless connection to imaging control unit 46 or the external processing system.

A light source 50 extends through passage 26 and is configured to emit light at distal portion 22 of elongated body 20. In example embodiments, passage 26 is configured to accommodate at least a portion of imaging device 40, e.g., signal transmission connection 45, and at least a portion of light source 50, e.g., a flexible optical conductor 52 operatively coupling, e.g., electrically coupling or optically coupling, light source 50 to imaging control device 46. In a particular embodiment, as mentioned above, elongated body 20 includes a first passage, e.g., first passage 26, configured to accommodate at least a portion of imaging device 40, e.g., signal transmission connection 45, and a second passage (not shown) configured to accommodate at least a portion of light source 50, e.g., flexible optical conductor 52. As shown in FIG. 3, in certain embodiments, imaging sensor 42 and/or light source 50 is positioned offset from longitudinal axis 28 of elongated body 20 to facilitate movement of other components, e.g., first catheter 14 and/or second catheter 16 along a length of elongated body 20. For example, in certain embodiments, imaging sensor 42 and/or light source 50 is positioned offset from longitudinal axis 28 to allow for deployment and retraction of a basket or a snare from a distal portion of second catheter 16.

In example embodiments, light source 50 includes any light source configured to emit a suitable amount of light at the target site. For example, light source 50 may include a light emitting diode (LED) light source, a fiber optic light source, a laser or another suitable light source. With elongated body 20 inserted into a patient's lumen, light source 50 emits one or more beams of optical energy, e.g., light, that propagates through a flexible optical conductor 52 of light source 50 extending through elongated body 20. Imaging device 40, e.g., imaging sensor 42, can image the illumination reflected by an object during navigation of imaging device 40 through the lumen or at the target site, e.g., interior walls of the lumen or kidney, in response to the beam of optical energy.

In example embodiments, image information captured and recorded by imaging device 40 is filtered and processed by imaging control unit 46 or another external processing system, having imaging software 54 for processing and displaying images on a display screen 56 positioned on imaging control unit 46 or an external display operatively coupled to imaging control unit 46 or the external processing system. In example embodiments, imaging control unit 46 or the external processing system controls light source 50 via optical conductor 52.

Referring further to FIGS. 5 and 6, in example embodiments, imaging scope 12 includes a flexible distal tip portion 60 coupled to distal portion 22 of elongated body 20. Flexible distal tip portion 60 has a second length less than the first length of elongated body 20. As shown in FIGS. 5 and 6, for example, imaging sensor 42 is disposed at, e.g., coupled to or at least partially embedded in, flexible distal tip portion 60. Referring further to FIGS. 5 and 6, imaging sensor 42 is disposed at flexible distal tip portion 60 and signal transmission connection 45 extends through passage 26 of elongated body 20 to operatively couple, e.g., communicatively couple, imaging sensor 42 to imaging control unit 46. In example embodiments, light source 50 is also disposed at, e.g., coupled to or at least partially embedded in, flexible distal tip portion 60. As shown in FIGS. 5 and 6, light source 50 and/or optical conductor 52 at least partially extend through passage 26 of elongated body 20 to operatively couple, e.g., communicatively couple, light source 50 to imaging control unit 46 or another external processing system. In example embodiments, flexible distal tip portion 60 is bulb-shaped, having an outer diameter equal to or greater than an outer diameter of elongated body 20.

In example embodiments, flexible distal tip portion 60 is configured to deflect in a plurality of directions including, for example, a first direction and a second direction different from the first direction. In certain embodiments, flexible distal tip portion 60 is configured to deflect at least 180°. Flexible distal tip portion 60 is configured to navigate through tight spaces and prevent perforation of the lumen or vessel in which imaging scope 12 is positioned. In example embodiments, flexible distal tip portion 60 has a length of 0.125 inch (0.318 cm) to 1.969 inch (5.00 cm) and, more particularly, a length of 0.787 inch (2.0 cm) to 1.0 inch (2.541 cm). Flexible distal tip portion 60 includes a suitable material and/or configuration to facilitate controllable steerability or deflection of flexible distal tip portion 60.

As shown in FIG. 5, in example embodiments, imaging scope 12 includes a proximal connector 62 coupled to proximal portion 24 of elongated body 20 opposite flexible distal tip portion 60. In example embodiments, proximal connector 62 has an outer surface with an outer diameter not great than, i.e., equal to or less than, the outer diameter of elongated body 20 such that other instruments or devices, e.g., first catheter 14 and/or second catheter 16, may move over proximal connector 62 and translate along the length of elongated body 20 with respect to distal portion 22, as described in greater detail below. In example embodiments, proximal portion 24 of imaging scope 12, e.g., proximal connector 62, has a low profile to allow the additional instruments or devices to be placed about imaging scope 12 at proximal portion 24.

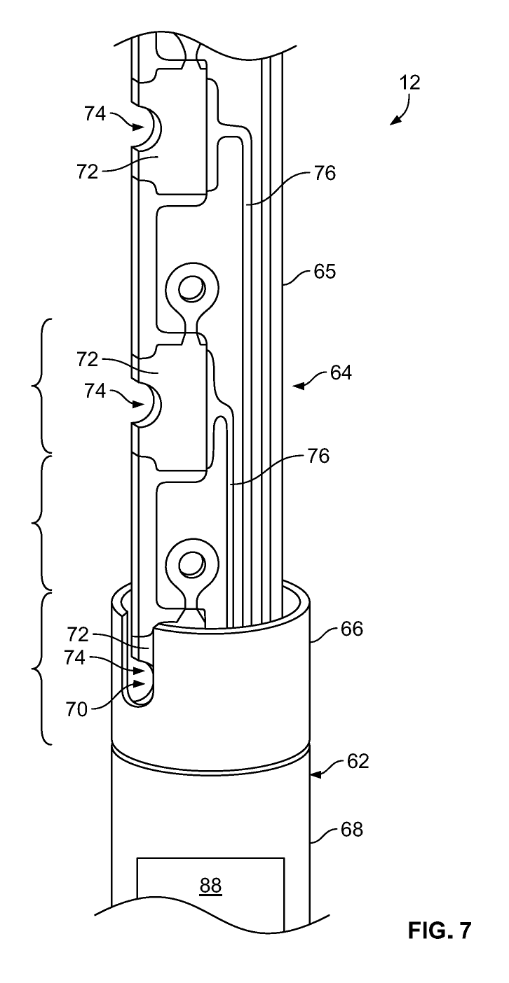

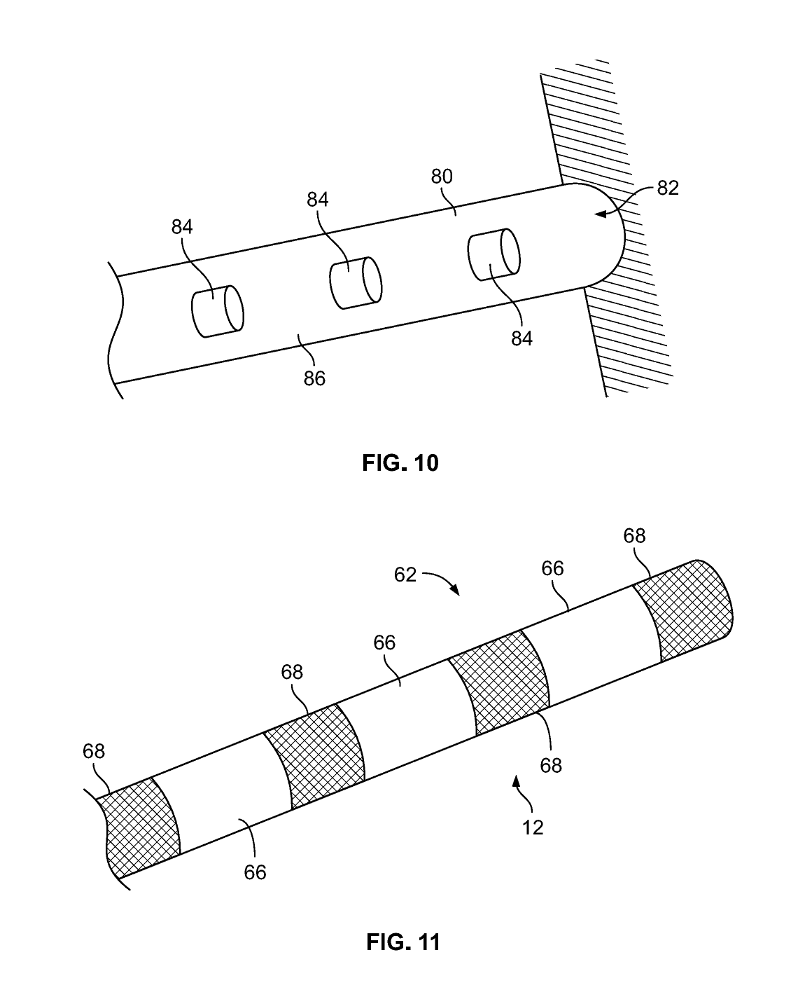

Referring now to FIGS. 7-11, in example embodiments, wiring harness 44 includes a proximal termination 64, e.g., a printed circuit board (PCB) 65, disposed at proximal connector 62 and operatively coupled, e.g., electrically coupled or optically coupled, to imaging device 40 and light source 50. In alternative embodiments, proximal termination 64 may include any suitable component or device configured to communicate with imaging device 40 and/or light source 50. In example embodiments, imaging sensor 42 is configured to detect image information and transmit one or more signals indicative of the detected image information to PCB 65 via signal transmission connection 45 extending through passage 26 to connect imaging sensor 42 to PCB 65. Light source 50 is disposed at flexible distal tip portion 22 and extends through passage 26 to electrically couple to PCB 65. In certain embodiments, PCB 65 is disposed within proximal connector 62. In example embodiments, one or more slip rings 66, e.g., a plurality of slip rings 66, forming at least a portion of proximal connector 62 are positioned about PCB 65, as shown in FIG. 7, for example. One or more annular insulator rings 68 are positioned adjacent a respective annular slip ring 66 and form a portion of proximal connector 62. For example, as shown in FIG. 11, proximal connector 62 includes a plurality of annular slip rings 66 alternating with a plurality of annular insulator rings 68 such that at least one annular insulator ring 68 is positioned between adjacent annular slip rings 66. Each annular slip ring 66 is electrically coupled to PCB 65 using a suitable coupling technique.

Referring to FIG. 7, in certain embodiments, each slip ring 66 includes a slot 70. PCB 65 includes one or more pads 72, e.g., a plurality of pads 72. Each pad 72 cooperates with a respective slip ring 66 to electrically couple imaging device 40 and/or light source 50 to PCB 65. In one embodiment, pad 72 forms a depression 74 that is aligned with slot 70 of a respective slip ring 66. With depression 74 aligned with cooperating slot 70, solder is received within slot 70 and depression 74 to electrically coupled slip ring 66 to pad 72 and PCB 65. As show in FIG. 7, each pad 72 is operatively coupled to a respective signal transmission line 76. Signal transmission line 76 electrically couples PCB 65 to imaging sensor 42 to facilitate signal transmission between imaging sensor 42 and PCB 65. Additionally or alternatively, signal transmission line 76 electrically couples PCB 65 to light source 50 to facilitate signal transmission between light source 50 and PCB 65.

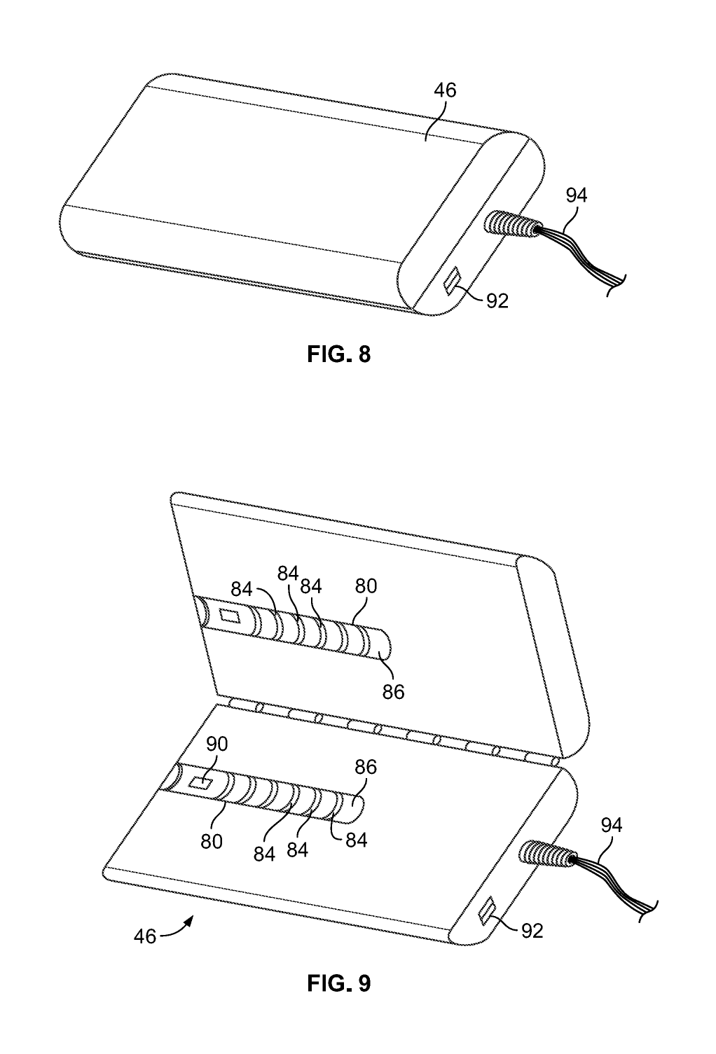

Referring now to FIGS. 8 and 9, in example embodiments, imaging control unit 46 is removably coupled to proximal connector 62. FIG. 8 is a perspective view of imaging control unit 46 in a closed configuration and FIG. 9 is a perspective view of imaging control unit 46 in an open configuration. As shown in FIG. 9, imaging control unit 46 includes two sections 80 that form a passage 82, shown in FIG. 11, with imaging control unit 46 in the closed configuration. In example embodiments, each section 80 includes one or more conductive brushes 84, e.g., a plurality of spaced conductive brushes 84, disposed on an inner surface 86 of section 80. Proximal connector 62 is positionable within passage 82 such that each annular ring 66 of proximal connector 62 contacts a respective conductive brush 84 in section 80 to electrically couple imaging control unit 46 to imaging scope 12 and, more particularly, electrically couple imaging control unit 46 to imaging device 40 and light source 50. In example embodiments, a window 88 is positioned in elongated body 20, e.g., proximal connector 62, as shown in FIG. 7, and a corresponding window 90 is positioned in passage 82, as shown in FIG. 9, to permit light to propagate between image control unit 40 and light source 50.

With imaging control unit 46 electrically coupled to proximal connector 62, imaging control unit 46 is in operational control communication with imaging device 40 to control operation of imaging device 40. Additionally, or alternatively, light source 50 extends through passage 26 to electrically couple to imaging control unit 46. With imaging control unit 46 electrically coupled to light source 50, imaging control unit 46 is in operational control communication with light source 50 to control operation of light source 50. In example embodiments, imaging control unit 46 includes a USB port 92 to electrically couple imaging control unit 46 to a remote processing system and an electrical connection 94 to electrically connect imaging control unit 46 to a standard electrical outlet.

Referring again to FIGS. 1 and 4, in example embodiments, first catheter 14, e.g., a steering or deflection catheter, includes a flexible distal tip portion 100 disposed at distal portion and a proximal portion 102 opposite flexible distal tip portion 100. In certain embodiments, first catheter 14 is rotatable about imaging scope 12. Referring further to FIGS. 12 and 13, a first lateral extension 104, such as a tab, a collar, a projection, a protuberance, or an appendage, for example, is disposed at flexible distal tip portion 100. First lateral extension 104 forms a second passage 106 having an inner diameter greater than the outer diameter of elongated body 20 and the outer diameter of proximal connector 62 of imaging scope 12 to allow imaging scope 12 to move within second passage 106. In certain embodiments, first lateral extension 104 also includes a longitudinal slit 110 intersecting second passage 106. In these embodiments, imaging scope 12 is positionable in second passage 106 and removable from second passage 106 through longitudinal slit 110 due to the elastic compliance of longitudinal slit 110. In example embodiments, first catheter 14 includes a second length that extends between flexible distal tip portion 100 and proximal portion 102. First catheter 14 includes at least one channel 112, such as channel 112 shown in FIG. 4, for example, which extends the second length of first catheter 14. In certain embodiments, channel 112 can be utilized to introduce additional instruments or devices to the target site, e.g., for suction, aspiration, and/or flushing.

As shown in FIGS. 1 and 4, for example, flexible distal tip portion 100 of first catheter 14 includes a knuckle or flexing area 114 configured to facilitate flexing of flexible distal tip portion 100 to manipulate multiple catheter system 10. In example embodiments, first catheter 14 includes a plurality of cables 116, e.g., three cables 116a, 116b, and 116c, equally spaced around a circumference of flexible distal tip portion 100. Cables 116 extend through channel 112 to proximal portion 102. As shown in FIG. 4, a suitable controller, such as a deflection actuator 118, is disposed at proximal portion 102 and operatively coupled to each cable 116 to independently control movement of cables 116 to control deflection of flexible distal tip portion 100 to direct first catheter 14 and multiple catheter system 10 in a desired direction. Deflection actuator 118 is operatively coupled to cables 116 to move flexible distal tip portion 100 in one of a plurality of directions, for example, a first direction or a second direction different from the first direction.

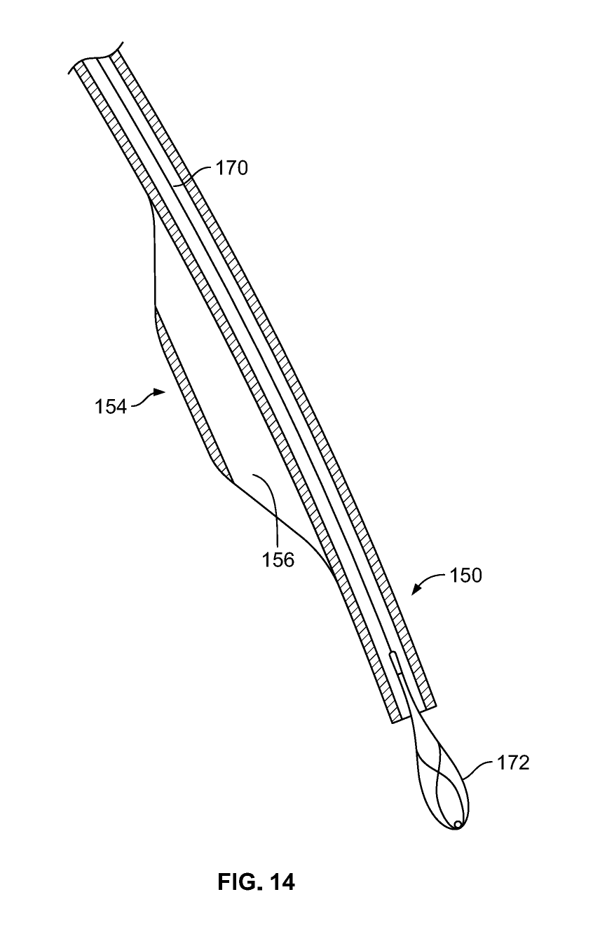

Referring again to FIGS. 1 and 4, in example embodiments, second catheter 16, e.g., a snare or basket catheter, includes a flexible distal tip portion 150 and a proximal portion 152 opposite flexible distal tip portion 150. In certain embodiments, second catheter 16 is rotatable about imaging scope 12. Referring further to FIG. 14, a second lateral extension 154 is disposed at flexible distal tip portion 150. In example embodiments, second lateral extension 154 is substantially identical or similar to first lateral extension 104. Second lateral extension 154 forming a third passage 156 having an inner diameter greater than the outer diameter of elongated body 20 and the outer diameter of proximal connector 62 of imaging scope 12 to allow imaging scope 12 to move within third passage 156. In certain embodiments, second lateral extension 154 also includes a longitudinal slit, e.g., similar to longitudinal slit 110 of first catheter 14 shown in FIG. 13, intersecting third passage 156. In these embodiments, imaging scope 12 is positionable in third passage 156 and removable from third passage 156 through the longitudinal slit. As shown in FIGS. 1 and 2, in example embodiments, second lateral extension 154 is disposed at flexible distal tip portion 150 of second catheter 16 proximally with respect to first lateral extension 104 disposed at flexible distal tip portion 100 of first catheter 14 to minimize the profile of multiple catheter system 10. Further, in example embodiments, first lateral extension 104 has a first profile and second lateral extension 154 has a second profile complementary with first profile of first lateral extension 104 to minimize a distance between first lateral extension 104 and second lateral extension 154 and further minimize an overall profile of multiple catheter system 10.

In example embodiments, second catheter 16 includes a third length that extends between flexible distal tip portion 150 and proximal portion 152. Second catheter 16 includes at least one channel, such as channel 170 shown in FIG. 14, for example, which extends the third length of second catheter 16. In certain embodiments, a basket 172 or snare, for example, is movably positioned in channel 170 and extendible from channel 170 at flexible distal tip portion 150. A suitable controller 174 is disposed at proximal portion 152 and operatively coupled to basket 172 to control operation of basket 172. In alternative embodiments, channel 170 is utilized to introduce additional instruments or devices to the target site, e.g., for suction, aspiration, and/or flushing, for example. In certain embodiments, second catheter 16 includes one or more of the following: a laser or a lithotripsy device for breaking and removing stones, for example.

Referring again to FIGS. 7-11, imaging control unit 46 is disposed at proximal portion 24 of elongated body 20 and operatively coupled to imaging device 40, e.g., coupled in operational controller communication with imaging device 40. Imaging control unit 46 is configured to control operation of imaging device 40 and, particularly imaging sensor 42, as well as light source 50. Imaging control unit 46 is communicatively coupled to imaging sensor 42 and configured to transmit signals to and receive signals from imaging sensor 42 via signal transmission connection 45, e.g., one or more signals indicative of imaging information detected by imaging sensor 42. Imaging control unit 46 is also configured to control the imaging detection of imaging sensor 42, e.g., a direction in which imaging sensor 42 is positioned. In certain embodiments, imaging control unit 46 is also configured to control operation of light source 50. For example, imaging control unit 46 may be configured to adjust parameters of the light emitted by light source 50, such as an amount or an intensity of the emitted light and/or a direction of the emitted light. In alternative embodiments, imaging sensor 42 and imaging control unit 46 may communicate using other suitable communication protocol including, for example, wireless communication. In certain embodiments, imaging control unit 46 is coupled to an external computer or processing system (not shown) for processing the imaging information that imaging control unit 46 receives from imaging sensor 42 through transmission connection 45 to generate one or more images of the target site.

In example embodiments, the imaging scope, coupled to first catheter 14 or independently, is placed transurethral, inserted through the patient's urethra and into the patient's bladder, for example. The imaging scope is navigated through the UVJ and into the ureter. As the imaging scope navigates up the ureter, the imaging scope visualizes the inner wall of the ureter and can be used diagnostically to identify strictures, stones, etc. Visualization, as well as the deflection properties of the flexible distal tip portion and the rigid core of the elongated body allows the imaging scope to navigate past the stricture, for example, and move up into the patient's kidney. If, for example, a stone in the ureter or the kidney needs to be removed, a rapid exchange second catheter is placed about the proximal portion of the imaging scope and inserted into the body lumen and up to the source of the stone burden. Devices or instruments for removing the stone are then introduced through the channel of the second catheter and visualized via the imaging scope.

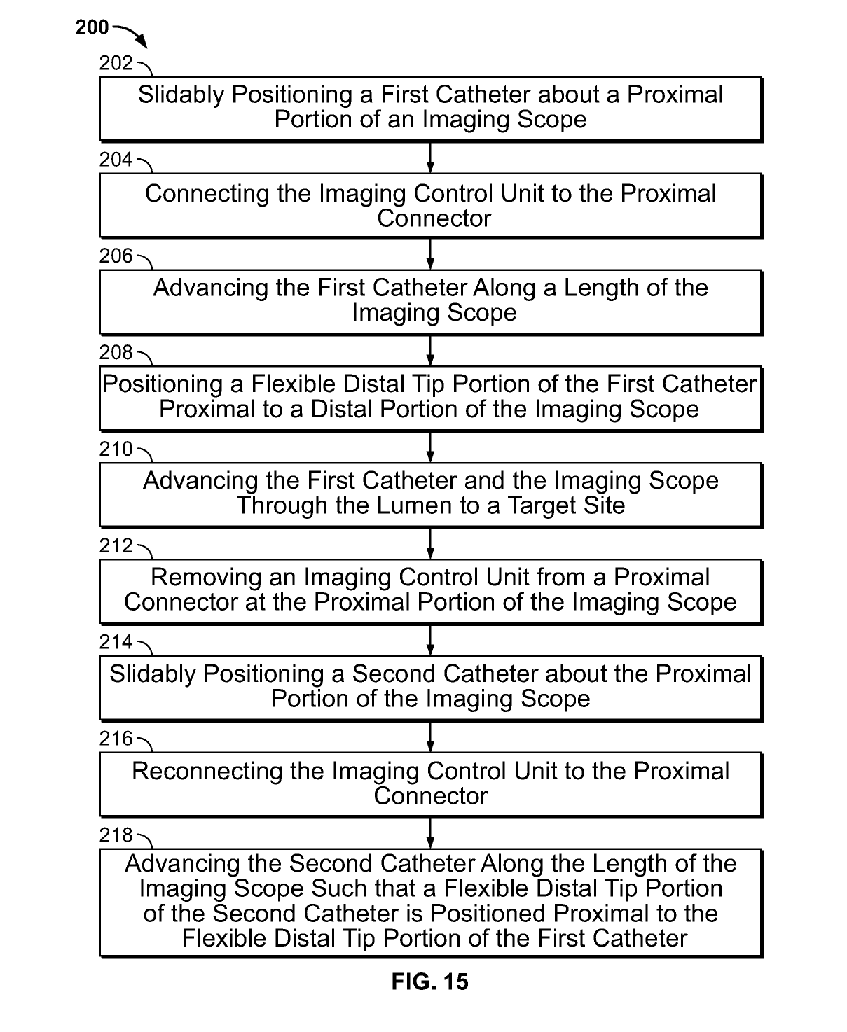

FIG. 15 is a flow diagram of an example method 200 for introducing an imaging scope, such as imaging scope 12 as shown in FIGS. 1-6, into a lumen of a patient. In step 202, a first catheter, e.g., a steering catheter, is slidably positioned about a proximal portion of the imaging scope and moveable along an elongated body of the imaging scope. In certain embodiments, an imaging control unit is removed from a proximal connector at the proximal portion of the imaging device to allow the first catheter to be slidably positioned about the proximal portion of the imaging scope. The imaging control unit is then connected 204 to the proximal connector to electrical couple the imaging control unit to an imaging sensor and a light source disposed at a distal portion of the imaging scope. The first catheter is advanced 206 along the elongated body of the imaging scope such that a flexible distal tip portion of the first catheter is positioned 208 immediately proximal to the distal portion of the imaging scope. The first catheter and the imaging scope is advanced 210 and moved up the patient's urethra, through the bladder, and into the appropriate ureter, utilizing the visualization aspect of the imaging scope and the steerability aspect of the first catheter. The imaging sensor can remain properly aligned within the lumen while the deflectable first catheter is rotated about the imaging scope to facilitate steering the multiple catheter system in a desired direction, e.g., left, right, up, or down.

When an occlusion or blockage is encountered and/or identified, the imaging control unit is removed 212 from the proximal connector at the proximal portion of the imaging device to allow a second catheter, e.g., a basket or snare catheter, to be slidably positioned 214 about the proximal portion of the imaging scope. The imaging control unit is then reconnected 216 to the proximal connector to electrical couple the imaging control unit to an imaging sensor and a light source disposed at a distal portion of the imaging scope. The second catheter is advanced 218 along the elongated body of the imaging scope such that a flexible distal tip portion of the second catheter is positioned immediately proximal to the flexible distal tip portion of the first catheter. The second catheter can then be utilized to remove the obstruction or blockage.

Imaging control unit 46 may be implemented as any of a number of different types of electronic devices. Some examples of imaging control unit 46 may include tablet computing devices, mobile devices, laptop and netbook computing devices or any other device capable of connecting with image device 40 and/or light source 50 and including a processor and memory for controlling image device 40 and/or light source 50 according to the techniques described herein.

In a very basic configuration, imaging control unit 46 includes, or accesses, components such as at least one control logic circuit, central processing unit, or processor, and one or more computer-readable media. Each processor may itself comprise one or more processors or processing cores. For example, each processor can be implemented as one or more microprocessors, microcomputers, microcontrollers, digital signal processors, central processing units, state machines, logic circuitries, fog computing, and/or any devices that manipulate signals based on operational instructions. In some cases, the processor may be one or more hardware processors and/or logic circuits of any suitable type specifically programmed or configured to execute the algorithms and processes described herein. The processor can be configured to fetch and execute computer-readable instructions stored in a computer-readable media or other computer-readable media.

Depending on the configuration of imaging control unit 46, computer-readable media may be an example of tangible non-transitory computer storage media and may include volatile and nonvolatile memory and/or removable and non-removable media implemented in any type of technology for storage of information such as computer-readable instructions, data structures, program modules or other data. The computer-readable media may include, but is not limited to, RAM, ROM, EEPROM, flash memory or other computer readable media technology, CD-ROM, digital versatile disks (DVD) or other optical storage, magnetic cassettes, magnetic tape, solid-state storage and/or magnetic disk storage. Further, in some cases, imaging control unit 46 may access external storage, such as RAID storage systems, storage arrays, network attached storage, storage area networks, cloud storage, or any other medium that can be used to store information and that can be accessed by the processor directly or through another computing device or network. Accordingly, computer-readable media may be computer storage media able to store instructions, modules or components that may be executed by the processor.

Computer-readable media may be used to store and maintain any number of functional components that are executable by the processor. In some implementations, these functional components comprise instructions or programs that are executable by the processor and that, when executed, implement operational logic for performing the actions attributed above to imaging control unit 46. Functional components of imaging control unit 46 stored in the computer-readable media may include the operating system and a user interface module for controlling and managing various functions of imaging device 40 and/or light source 50, and for generating one or more user interfaces on imaging control unit 46.

Imaging control unit 46 may further include one or more communication interfaces, which may support both wired and wireless connection to various networks, such as cellular networks, radio, Wi-Fi networks, close-range wireless connections, near-field connections, infrared signals, local area networks, wide area networks, the Internet, and so forth. The communication interfaces may further allow a user to access storage on or through another device, such as a remote computing device, a network attached storage device, cloud storage, or the like.

Imaging control unit 46 may further be equipped with one or more various input/output (I/O) components. Such I/O components may include a touchscreen and various user controls (e.g., buttons, a joystick, a keyboard, a keypad, etc.), a haptic or tactile output device, connection ports, physical condition sensors, edge devices, and so forth. For example, the operating system of imaging control unit 46 may include suitable drivers configured to accept input from a keypad, keyboard, or other user controls and devices included as I/O components. Additionally, imaging control unit 46 may include various other components that are not shown, examples of which include removable storage, a power source, such as a battery and power control unit, a PC Card component, and so forth.

Various instructions, methods and techniques described herein may be considered in the general context of computer-executable instructions, such as program modules stored on computer storage media and executed by the processors herein. Generally, program modules include routines, programs, objects, components, data structures, etc., for performing particular tasks or implementing particular abstract data types. These program modules, and the like, may be executed as native code or may be downloaded and executed, such as in a virtual machine or other just-in-time compilation execution environment. Typically, the functionality of the program modules may be combined or distributed as desired in various implementations. An implementation of these modules and techniques may be stored on computer storage media or transmitted across some form of communication.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims.

One skilled in the art will realize that a virtually unlimited number of variations to the above descriptions are possible, and that the examples and the accompanying figures are merely to illustrate one or more examples of implementations.

It will be understood by those skilled in the art that various other modifications can be made, and equivalents can be substituted, without departing from claimed subject matter. Additionally, many modifications can be made to adapt a particular situation to the teachings of claimed subject matter without departing from the central concept described herein. Therefore, it is intended that claimed subject matter not be limited to the particular embodiments disclosed, but that such claimed subject matter can also include all embodiments falling within the scope of the appended claims, and equivalents thereof.

In the detailed description above, numerous specific details are set forth to provide a thorough understanding of claimed subject matter. However, it will be understood by those skilled in the art that claimed subject matter can be practiced without these specific details. In other instances, methods, devices, or systems that would be known by one of ordinary skill have not been described in detail so as not to obscure claimed subject matter.

Reference throughout this specification to “one embodiment” or “an embodiment” can mean that a particular feature, structure, or characteristic described in connection with a particular embodiment can be included in at least one embodiment of claimed subject matter. Thus, appearances of the phrase “in one embodiment” or “an embodiment” in various places throughout this specification are not necessarily intended to refer to the same embodiment or to any one particular embodiment described. Furthermore, it is to be understood that particular features, structures, or characteristics described can be combined in various ways in one or more embodiments. In general, of course, these and other issues can vary with the particular context of usage. Therefore, the particular context of the description or the usage of these terms can provide helpful guidance regarding inferences to be drawn for that context.

What is claimed is:

1. An imaging scope for a multiple catheter system, the imaging scope comprising:
an elongated body having a first length, the elongated body including a passage extending along the first length;
a flexible distal tip portion coupled to the elongated body;
an imaging device including a wiring harness at least partially disposed in the passage and an imaging sensor disposed at the flexible distal tip portion, the imaging sensor operatively coupled to the wiring harness; and
a proximal portion coupled to the elongated body opposite the flexible distal tip portion, the proximal portion having an outer surface, the proximal portion comprising:
a proximal termination of the wiring harness; and
an annular slip ring electrically coupled to the proximal termination.

2. The imaging scope of claim 1, wherein the imaging sensor is configured to detect image information and transmit one or more signals indicative of the detected image information to the proximal termination.

3. The imaging scope of claim 1, wherein the wiring harness comprises a signal transmission connection extending through the passage to connect the imaging sensor to the proximal termination.

4. The imaging scope of claim 1, wherein the proximal portion further comprises an annular insulator ring adjacent the annular slip ring.

5. The imaging scope of claim 1, further comprising an imaging control unit electrically coupled to the proximal portion, wherein the imaging control unit comprises a passage with a conductive brush disposed in the passage, and the proximal portion is positionable within the passage such that the annular ring contacts the conductive brush to electrically couple the imaging control unit to the proximal portion.

6. The imaging scope of claim 5, wherein the imaging control unit is removably coupled to the proximal portion.

7. The imaging scope of claim 5, wherein the imaging control unit is in operational control communication with the imaging device to control operation of the imaging device.

8. The imaging scope of claim 5, further comprising a light source disposed at the flexible distal tip portion, the light source extending through the passage and operatively coupled to the imaging control unit.

9. The imaging scope of claim 8, wherein the imaging control unit is in operational control communication with the light source to control operation of the light source.

10. The imaging scope of claim 5, wherein the imaging control unit comprises a USB port to electrically couple the imaging control unit to a remote processing system.

11. The imaging scope of claim 1, wherein the slip ring includes a slot and the proximal termination comprises printed circuit board (PCB), the PCB including a pad having a depression aligned with the slot, the slot and the depression for receiving solder to electrically couple the slip ring to the PCB.

12. The imaging scope of claim 11, further comprising a signal transmission line electrically coupling the PCB to the imaging sensor.

13. The imaging scope of claim 1, further comprising a light source disposed at the flexible distal tip portion, the light source extending through the passage and electrically coupled to the proximal termination.

14. The imaging scope of claim 1, wherein the flexible distal tip portion has an outer diameter greater than an outer diameter of the elongated body.

15. The imaging scope of claim 1, wherein the proximal portion has an outer diameter not greater than an outer diameter of the elongated body to allow a device to be placed about the imaging scope at the proximal portion, the device movable along the elongated body.

16. A multiple catheter system, comprising:
an imaging scope including:
an elongated body having a first length and a first outer diameter, the elongated body including a first passage extending along the first length;
a flexible distal tip portion coupled to the elongated body, the flexible distal tip portion including an imaging sensor and a light source; and
a proximal connector coupled to the elongated body opposite the flexible distal tip portion and operatively coupled to the imaging sensor and the light source, the proximal connector having a second outer diameter not greater than the first outer diameter;
a first catheter comprising:
a flexible distal tip portion; and
a first lateral extension disposed at the flexible distal tip portion, the first lateral extension forming a second passage having an inner diameter greater than the first outer diameter and the second outer diameter, wherein the imaging scope is movably positionable within the second passage.

17. The multiple catheter system of claim 16, wherein the first lateral extension further comprises a longitudinal slit intersecting the second passage and the imaging scope is positionable in the second passage and removable from the second passage through the longitudinal slit.

18. The multiple catheter system of claim 16, wherein the flexible distal tip portion of the first catheter comprises a flexing area configured to facilitate flexing of the flexible distal tip portion to manipulate the multiple catheter system.

19. The multiple catheter system of claim 16, wherein the first catheter further comprising:
a proximal portion opposite the flexible distal tip portion, the first catheter having a second length between the proximal portion and the flexible distal tip portion;
a channel extending along the second length;
a plurality of cables equally spaced around a circumference of the flexible distal tip portion of the first catheter, the plurality of cables extending through the channel to the proximal portion; and
a deflection actuator disposed at the proximal portion, the deflection actuator coupled to each of the plurality of cables to control deflection of the flexible distal tip portion.

20. The multiple catheter system of claim 16, wherein the first catheter is rotatable about the imaging scope.

21. The multiple catheter system of claim 16, further comprising a second catheter, the second catheter comprising:
a flexible distal tip portion; and
a second lateral extension disposed at the flexible distal tip portion, the second lateral extension forming a third passage having an inner diameter greater than the first outer diameter and the second outer diameter, wherein the imaging scope is movably positionable within the third passage.

22. The multiple catheter system of claim 21, wherein the second lateral extension is disposed at the flexible distal tip portion of the second catheter proximally with respect to the first lateral extension disposed at the flexible distal tip portion of the first catheter.

23. The multiple catheter system of claim 21, wherein the second catheter further comprises:
a proximal portion opposite the flexible distal tip portion, the second catheter having a second length between the proximal portion and the flexible distal tip portion;
a channel extending between the flexible distal tip portion and the proximal portion;
a basket movably positioned in the channel and extendible from the channel at the flexible distal tip portion; and
a controller disposed at the proximal portion, the controller operatively coupled to the basket to control operation of the basket.

24. The multiple catheter system of claim 23, wherein the second catheter comprises one or more of the following: a laser or a lithotripsy device.

25. The multiple catheter system of claim 21, wherein the first lateral extension has a first profile and the second lateral extension has a second profile complementary with the first profile to minimize a distance between the first lateral extension and the second lateral extension and minimize a profile of the multiple catheter system.

26. A method for introducing an imaging scope into a lumen of a human, the method comprising:
slidably positioning a first catheter about a proximal portion of an imaging scope;
advancing the first catheter along a length of the imaging scope;
positioning a flexible distal tip portion of the first catheter proximal to a distal portion of the imaging scope; and
advancing the first catheter and the imaging scope through the lumen to a target site.

27. The method of claim 26, further comprising:
removing an imaging control unit from a proximal connector at the proximal portion of the imaging scope;
slidably positioning a second catheter about the proximal portion of the imaging scope; and
advancing the second catheter along the length of the imaging scope such that a flexible distal tip portion of the second catheter is positioned proximal to the flexible distal tip portion of the first catheter.

28. The method of claim 26, further comprising:
removing an imaging control unit from a proximal connector at the proximal portion of the imaging device to allow the first catheter to be slidably positioned about the proximal portion of the imaging scope; and
reconnecting the imaging control unit to the proximal connector to electrical couple the imaging control unit to an imaging sensor and a light source disposed at a distal portion of the imaging scope.
